(19) United States
(12) Patent Application Publication
(10) Pub. No.: US 2005/0112251 A1
(43) Pub. Date: May 26, 2005

(54) Preservation of liquids

(76) Inventors: Jacobus Stark, ZM Rotterdam (NL); Nicole Liliane Dutreux, HJ Schiedam (NL)

Correspondence Address: MORRISON & FOERSTER LLP, 3811 VALLEY CENTRE DRIVE, SUITE 500, SAN DIEGO, CA, 92130-2332, US

(21) Appl. No.: 10/505,605

(22) PCT Filed: Feb. 25, 2003
(86) PCT No.: PCT/EP03/01922

(30) Foreign Application Priority Data
Feb. 25, 2002 (EP) 02075929.6

(51) Int. Cl. C12C 1/00

(52) U.S. Cl. 426/330.3

(57) ABSTRACT

The present invention relates to a liquid which have been treated with an electroporation method and which comprises an effective amount of our antifungal compound.

### FIELD OF THE INVENTION

The present invention relates to a process for preserving liquids, e.g. beverages, against spoilage by fungi.

### BACKGROUND OF THE INVENTION

The prevention of fungus growth is an important topic to the food industry. Fungal spoilage can lead to serious economic losses. Some food products are very susceptible to fungal growth. Beverages such as fruit juices, lemonades, wine, ice tea and beer are examples of such food products. Spoilage by fungi does not only affect the quality of the product, but also represents a health risk. It is well known that some fungus species, which grow in beverages, can produce mycotoxins. Some mycotoxins are extremely dangerous because they can induce cancer. Therefore the presence of unwanted fungus species in food products should always be prevented.

Up to now microorganisms present in food products, such as beverages, are mostly eliminated by a heat treatment, e.g. pasteurization or sterilization. However such treatment affects the quality of the product. Chemical and physical properties of the product will change in a negative way causing loss of nutritional value, organoleptic properties and colour amongst others. No methods of preventing spoilage of food products, especially by fungus species, without affecting the quality of the food product in a negative way are presently known.

### DESCRIPTION OF THE INVENTION

The object of this invention is to provide microbially safe liquid food products without alteration of its original characteristics, such as colour, texture, flavour, odour and nutritional value. According to the invention the liquid foodstuff is treated with an electroporation method, such as Pulsed Electric Field, and an antifungal agent which prevents spoilage of said products by fungi, is added to the liquid foodstuff. Surprisingly we found that the pulsed electric field process is effective against vegetative cells and the antifungal composition prevents spoilage caused by germination and outgrowth of fungal spores. Preservatives, which prevent the outgrowth of bacterial spores, may also be added. The process of the invention is conducted at minimal elevated temperatures and has an antimicrobial effect against spoilage organisms, including heat-resistant fungi, without affecting the sensory and nutritional characteristics of the liquid.

The present invention provides a liquid which has been treated with an electroporation method and which comprises an effective amount of an antifungal compound, an electroporated liquid comprising an effective amount of an antifungal compound, and/or a liquid which is microbially stable for at least 30 days and is substantially free of sensory effect induced by a heat treatment or has a minimal sensory effect compared with the effect induced by heat treatment, and containing an amount of antifungal agent.

The quality of PEF-treated product depends on the inactivation level of the target micro-organism(s) and on the sensorial and nutrional properties which mainly depend on the temperature reached during the treatment.

A PEF-treatment is called a mild-treatment when the temperature reached during the PER treatment stays below the temperature that induces changes of the product properties, or below the temperature usually attained by conventional heat treatment (pasteurisation).

A PEF-treatment or an antifungal compound can induce total inactivation of the target microorganism(s) or sublethal damage (partial inactivation, destabilisation of the microbial structure). However a PEF-treatment will in general not inactivate spores of microorganisms. A sub optimal PEF-treatment or a sub optimal natamycin concentration induce sublethal damage. For example a sub optimal natamycin concentration is a natamycin concentration lower than the MIC value (Mnimal Inhibition Concentration).

Suboptimal treatments have sensorial (minimal PEF-temperature) and economic (lower PEF-energy and reduced natamycin concentration) benefits.

For example we have found that orange juice treated with the process of the invention during storage at 4° C. retains more vitamin C than heat-treated juice. Moreover, the juice treated according to the invention has a lower browning index than heat-treated juice. PEF-treated juice showed a brighter color that heat-treated juice. And finally the particles of juice treated according to the invention have smaller particles than heat-treated orange juice. In general the process of the invention shows several advantages compared to heat treatment for example in the better retention of Vitamin C, prevention of unwanted maillard reactions and no noticeable differences between liquids treated with the process of the present invention and untreated liquids. These differences are much smaller than differences between heat treated (pasteurised) liquids and untreated liquids.

The present invention further provides a method of treating a liquid, which comprises:
(a) subjecting the liquid to an electroporation method; and
(b) adding an effective amount of an antifungal compound to the liquid.

The method described in the present invention can be used to prevent the growth of microorganisms, especially fungi, in a wide variety of liquid products.

An electroporation method is advantageously used in combination with the addition of an antifungal compound to preserve a liquid.

Examples of an electroporation method, which can be used as non-thermal preservation method for liquid food products, are methods using electricity, such as methods known under the name Pulsed Electric Field (PEF), High Pulsed Electric Field, Low Pulsed Electric Field, High Voltage Arc Discharge and Streamer Plasma. The present invention may utilise any of these methods, which induce electroporation of the membrane of microorganisms. The preferred electroporation method is PEF.

PEF can be applied to fruit juices, lemonades, wine, beer, liquid egg products and other types of pumpable products.

PEF processing involves the application of pulses of high or low voltage to products placed between two electrodes. The shortness of the pulses attempts to minimize heating of the product. The pulsed electric field device for the treatment of pumpable food products has at least two electrodes for supplying an electric field to the liquid product. All electrodes include an electrode flow chamber for accepting the flow of the liquid food product and for making electric contact with the liquid product. The PEF treatment device also includes at least one insulator positioned between two consecutive electrodes in order to electrically insulate these two electrodes from each other. The electrode flow chambers and the insulator flow chamber(s) include an inlet aperture and an outlet aperture. The PEF apparatus may also include a voltage pulse generator for supplying a pulsed electric field of high or low intensity, a PEF liquid product treatment device for subjecting the liquid product to the pulsed electric field and all required annexes such as a tank for storing the product to be treated, a tank to receive the treated product, a device for removing oxygen and other gas from the liquid, a pump for providing a continuous flow and at least one heat exchanger for regulating the temperature of the liquid product. The PEF treatment device is in communication with the high voltage pulse generator that applies a high voltage signal of variable voltage, frequency, pulse duration, shape and polarity to the electrodes.

The high voltage electric field causes death of the vegetative cells of microorganisms by electroporation or lysis of the microbial cell membrane. Pulsed electric fields treatments are effective at inactivating vegetative cells. We have found that fungus spores will survive the PEF treatment because of the spore's rigid structure and ability to resist unfavourable environmental conditions. As a result, PEF treatment alone can not be used in practice to prevent spoilage of liquids in which fungi can grow. PEF treatment will not prevent the outgrowth of fungal spores, whose occurrence and growth of fungus spores in beverages represents an important problem in the current food industry. More recently, the occurrence of more heat resistant fungus species has caused problems in the food industry.

Several antifungal agents, which are used to prevent outgrowth of fungi in food products, can be used in the process of the present invention. Examples of fungicides are polyene antimycotics (e.g. natamycin, nystatin, lucensomycin or amphotericin B); organic acids (e.g. benzoic acid, sorbic acid, propionic acid and lactic acid); salts of said organic acids (e.g. benzoate, sorbate, propionate and lactate); imidazoles or their salts (e.g. imazalil); or any antifungal agent known in the art. The antifungal composition can also be a combination of two or more of the above-mentioned compounds. The effective amount of an antifungal together with the electroporation method prevents the growth of micro-organisms, especially fungus, in the electroporated liquid product. An effective amount of an antifungal compound can be a suboptimal concentration, the MIC value, or higher.

A sub optimal concentration is preferred.

The present invention discloses the combination of the use of an antifungal composition and together with methods based on the electroporation of the membrane of microorganisms (such as Pulsed Electric Field) to prevent the spoilage of liquids by fungi.

Unexpectedly, we have found that spores of fungus species, especially heat resistant fungus species, are not affected completely by a PEF treatment. A PEF treatment alone even leads to specific selection of these spoilage and/or pathogenic species in food products.

Moreover, we have also found that antifungal agents, especially natamycin and sorbate are not inactivated by the PEF treatment. Therefore said agents can be added to the liquid product, e.g. a fruit juice, before executing the PEF treatment. Alternatively the antifungal agent may also be added to the liquid after the PEF treatment. The PEF treatment will inactivate all vegetative cells present in the product. However fungus spores will not be inactivated. The antifungal agent will prevent spoilage caused by the germination of fungus spores in the liquid. In addition, an antibacterial agent can be added to the product to inactivate bacterial spores. Preferably nisin or lysozyme are used as antibacterial agent.

A preferred fungicide to prevent spoilage of food products by fungi is natamycin. Natamycin has been used for more than 30 years to prevent outgrowth of fungi on cheeses and sausages. Natamycin is on the market under the brand name of Delvocid® or Actistab®), a powder composition containing 50% (w/w) of natamycin and 50% (w/w) of lactose or glucose respectively. Natamycin has a MIC (Minimal Inhibition Concentration) of less than 10 ppm for most food born fungi while its solubility in water is from 30 to 50 ppm. Natamycin can easily be applied to prevent spoilage by fungi in beverages by mixing the powder through the liquid. Under normal hygienic conditions for beverages such as fruit juices, wine, beer, ice tea and lemonades a concentration of 1-50 ppm, preferably 3-10 ppm of natamycin is usually sufficient to prevent fungal growth. The effective amount of antifungal compound means the amount of the antifungal compound needed to prevent fungus growth. A method for determining the minimal effective amount of the antifungal compound is described in Example 1. We have found that natamycin is especially effective against the growth of heat resistant fungus species.

Another preservative that prevents fungus growth is sorbate/sorbicacid. Usually concentrations of 500-2000 ppm of sorbate are sufficient to prevent fungal growth. However, said concentrations will sometimes be insufficient to prevent spoilage of the product because sorbate resistant fungal species may be present. In such cases, higher concentrations are used.

The concentration of the antifungal agent, e.g. natamycin or sorbate can be reduced considerably by combined treatment with PEF. Although of course the exact concentration of the fungicide is determined by many external factors, such as the hygienic conditions, a reduction of at least 50% of the necessary concentration of the antifungal agent can be achieved.

This invention describes for the first time a method to inactivate all microorganisms, especially fungi, present in a liquid without affecting the organoleptic properties, the colour and the nutritional value of the product in a negative way.

The present method is suitable for the treatment of consumable liquids such as fruit juices, wine, beer, lemonades, ice tea, liquid eggs, milk products, desserts and yoghurts. Therefore the consumable liquids are liquids preferably comprising sugars and/or proteins and/or free amino acids. Moreover the present invention can also be used for the treatment of other pumpable liquids such as processing fluid streams, blood, water, ecosystem waters, pharmaceutical products, cosmetics and process water. The term “pumpable products” means any product, which is capable of being pumped or conveyed through pipes or conduits, including solid items conveyed in a conductive aqueous solution. Examples are products obtained from fruits, vegetables and milk such as marmalades, jams, fruit pulp, vegetable extracts, oil, fluid butter and mayonnaise. Alternatively the fluid may also contain pieces of fruit. A heat treatment, e.g. pasteurisation or sterilization of consumable products containing sugars and proteins and/or free amino acids, will result in a change in a negative way of the physical properties which is prevented when PEF-treatment is used instead of the heat treatment. For example Maillard reactions and Maillard reaction products are prevented.

The invention described herein is related to any method based on the electroporation of membranes of microbial cells (such as PEF) in combination with an antifungal compound or combinations of antifungal compounds. Optionally antibacterial agents known in the art may be added. Preferred antibacterial agents used are nisin and lysozyme.

The process of the invention can be performed with any pulsed electric field apparatus independently of the nature of the individual physical components of the PEF device, such as pipes, wire, switches, power supplies, pulser, sensors and computers. Further the process of the invention can be used in any pulsed electric field preservation process, independently of the intensity of the different parameters, such as the electric field, the frequency, the pulse length, the number of pulses, the pulse shape and polarity and the total energy density applied.

The process of this invention is specifically suitable for inhibition of growth of heat-resistant fungi without causing detrimental effects to the product. Spoilage of beverages, thermally processed fruits and fruit products by heat-resistant fungi has been recognized (Tournas, V. (1994), Heat-resistant fungi of importance to the food and beverage industry, Critical Review for Microbiology, 20, 243-263 and Beuchat, L. R., Rice, S. L. (1979), Bysschlamys spp. and their importance in processed fruits, Advances in Food Reseach, 25, 237-288). Byssochlamys fulva, Byssochlamys nivea, Talaromyces macrosporus have been most frequently encountered. Heat-resistant fungi are characterized by the production of ascospores or similar structures with heat resistance. This enables them to survive the thermal processes given to some beverages. Production of pectic enzymes by Byssochlamys can result in complete breakdown of texture in fruit products and also can result in off-flavor development. Some Byssochlamys species produce patulin and byssochlamys acid, which both have toxic effects. Heat-resistant fungi, therefore, constitute a public hazard as well as a spoilage problem.

While ascospores of fungi are not inactivated in general by pulsed electric field, addition of an antifungal agent, such as natamycin, to the product to be processed or after processing prevents growth of fungi including heat-resistant fungi.

### EXAMPLE 1

### Natamycin MIC-Value of Heat Resistant Spoilage Fungus Species

This example demonstrates the antifungal effect of natamycin against several important spoilage fungi responsible for many problems in todays food industry.

The minimal inhibition concentration (MIC) of these fungi or the minimal effective amount of the antifungal compound was determined using the agar diffusion method, which is well known in the art. Fungus spores were grown on agar plates containing different concentrations of natamycin. The concentration of natamycin on which no visible growth could be observed was considered as the minimal inhibition concentration for that particular fungus strain. The results are presented in Table 1. The results clearly demonstrate that natamycin at concentrations <5 ppm inhibits the outgrowth of fungal spores which survived the PEF treatment.

TABLE 1
sensitivity of spoilage fungi towards natamycin

| Fungus Species | MIC (ppm) | Isolated from |
|---|---|---|
| Byssochlamys nivea 424.300 | <2.5 | — |
| Mucor circinelloides 366.70 | <2.5 | Strawberries |
| Aspergillus versicolor 245.62 | <2.5 | — |
| Stemphiliomma valparadisiacum 510.83 | <5 | Apple juice |
| Talaromyces macrosporus 130.89 | <2.5 | Pineapple juice |
| Cladosporium tenuissimum 117.79 | <2.5 | Fruit |
| Zygosporium mycophilum 396.49 | <2.5 | Apple pulp |
| Peacilomyces variotii 10274 | <2.5 | — |

### EXAMPLE 2

### The Antifungal Effect of Natamycin in Apple Juice

This example describes the activity of natamycin against two fungal species, which are well known for causing spoilage problems in food industry.

All experiments were done with pure apple juice. The chosen concentrations of natamycin were sub-optimal (=below MIC).

At higher concentrations, natamycin fully inhibits the outgrowth of said strains.

All experiments were executed in duplo.
a. The effect of natamycin against the spore forming yeast Zygosaccharomyces bailii CBS 1097 was examined. Apple juice was inoculated with 10⁴ Colony Forming Units (CFU)/ml of apple juice. Natamycin was added at a concentration of 1 ppm.
b. The effect of natamycin was tested against Penicillium italicum ATCC 36041. A spore suspension was prepared using well-known methods. The freshly prepared spore suspension was added to apple juice to a final concentration of 104 spores/ml of apple juice. Natamycin was added at a concentration of 5 ppm.

The samples were incubated at room temperature until fungal growth was visually observed (turbidity of the juice). The results are presented in table 2.

TABLE 2
Activity of natamycin under sub-optimal conditions

| | Natamycin | Time to |
|---|---|---|
| Microorganism | Concentration | spoilage |
| a. Zygosaccharomyces bailii | 1.0 ppm | 2 days |
| b. Penicillium italicum | 5.0 ppm | 4 days |

### EXAMPLE 3

### The antifungal effect of a Pulsed Electric Field (PEF) treatment In apple juice

This example describes the effect of a Pulsed Electric Field treatment against the two fungal strains described in example 2.

The chosen PEF-treatment parameters were sub-optimal. At higher energy levels the outgrowth of said strains is fully inhibited.

The pulsed electric fields treatments were applied according to well-known procedures for PEF-treatments of liquids. The apple juice to be treated was pumped through the PEF-treatment chamber with a flow rate of 60 litres per hour. The treatment temperature also called juice temperature before the PEF-treatment chamber was 17° C. The value of the electric field strength and the pulse length kept constant were 35 kV/cm and 2-μs, respectively. Three samples points were validated by variation of the pulse frequency, from 8.6 Hz (treatment A) to 5.7 Hz (treatment B) and 2.9 Hz (treatment C). With the above-mentioned parameters, the apple juice received 12, 8 or 4 pulses; those correspond to an energy density of 71, 37 and 12.5 J/ml of juice, respectively. In these conditions, the temperature of the apple juice did not exceed 34° C., which implies a mild treatment.

All experiments were executed in duplo.
a. A PEF-treatment was applied on Zygosaccharomyces bailii at a concentration of 104 CFU/ml of apple juice.
b. A PEF-treatment was applied on Penicillium italicum at a concentration of 104 spores/ml.

The results are presented in table 3.

TABLE 3
Activity of PEF under sub-optimal conditions

| | Time to spoilage | Time to spoilage |
|---|---|---|
| Microorganism | Treatment A | Treatment B & C |
| a. Z. bailii | 4 days | 2 days |
| b. P. italicum | 6 days | 6 days |

### EXAMPLE 4

### The Antifungal Effect of a Combined Treatment: PEF with Natamycin In Apple Juice

This example describes the effect of natamycin combined with a Pulsed Electric Field treatment against the fungal strains described in example 2.

The chosen inoculation levels, natamycin concentrations and PEF treatments were as described in the examples 2 and 3. The natamycin can be added before or after the PEF treatment.

All samples were incubated at room temperature until fungal growth was visually observed (turbidity of the juice). The results are presented in table 4.

TABLE 4
Activity of the combination of natamycin and PEF

| | | Time to spoilage | |
|---|---|---|---|
| | | Natamycin added | Natamycin added |
| | Treatment | after | before PEF- |
| Microorganism | conditions | PEF-treatment | treatment |
| a. Z. bailli | PEF-treatment A | not determined | >30 days |
| | PEF-treatment B | >30 days | not determined |
| b. P. italicum | PEF-treatment C | >30 days | >30 days |

The above results clearly demonstrate that a combination of low natamycin concentrations and an extremely mild PEF-treatment prevents the spoilage of apple juice. In particular, the data demonstrate that the combined effect is substantially greater than the effect of natamycin or PEF alone.

This example discloses for the first time the production of microbial stable apple juice with hardly any loss of organoleptic or nutritional properties.

### EXAMPLE 5

### Stability of Natamycin in Apple Juice during PEF Treatment

This example illustrates the resistance of natamycin to PEF treatments. Natamycin was added to apple juice at a concentration of 5 ppm and submitted to pulsed electric field treatments as described in example 3. The natamycin content after the PEF-treatments was determined by HPLC. None of the analyzed samples showed a decrease in the natamycin concentration.

### EXAMPLE 6

### The Effect of Sorbate Against a Sorbate-Resistant Yeast in Apple Juice

This example describes the activity of sorbate against the sorbate-resistant yeast Zygosaccharomyces bailii CBS 1097.

Zygosaccharomyces bailii was inoculated in apple juice as described in example 2. Sorbate was added at a concentration of 800 ppm. The samples were incubated at room temperature until fungal growth was visually observed (turbidity of the juice), which was the case after 6 days of incubation.

All experiments were executed in duplo.

### EXAMPLE 7

### The Antifungal Effect of a Combined Treatment: PEF with Sorbate in Apple Juice

This example describes the effect of sorbate combined with a Pulsed Electric Field treatment against the sorbate-resistant yeast Zygosaccharomyces bailii as described in example 6.

The PEF-treatment was used as described in the example 3. The treatment parameters were chosen so that the juice temperature did not exceed 26° C. (treatment B).

The samples were incubated at room temperature. Fungal growth was not observed for at least 30 days.

It can be concluded that as for natamycin (example 4) also for sorbate in combination with a PEF treatment microbialy stable apple juice without hardly any loss of quality can be prepared.

### EXAMPLE 8

### The Antifungal Effect of Natamycin Against Heat-Resistant Fungus Spores in Apple Juice

This example describes the activity of natamycin against spores of a heat resistant fungal species, which is well known for causing spoilage problems in food industry. All experiments were executed on Talaromyces macrosporus CBS 130.89 spores suspended in pure apple juice. The chosen concentration of natamycin was sub-optimal. At higher concentrations natamycin fully inhibits the outgrowth of the strain. All experiment were executed in duplo.

Apple juice was inoculated with 104 spores/ml of apple juice. Natamycin was added at a concentration of 2.5 ppm. The spores suspended in the apple juice were submitted to a heat treatment at 80° C. for 5 minutes in order to stimulate their germination.

The samples were incubated at room temperature until fungal growth was visually observed (turbidity of the juice). Growth was observed after 4 days of incubation at 25° C.

All experiments were executed in duplo.

### EXAMPLE 9

### The Antifungal Effect of a Pulsed Electric Field (PEF) Treatment Against Heat-Resistant Fungus Spores in Apple Juice

This example describes the effect of a Pulsed Electric Field treatment against heat-resistant spores of Talaromyces macrosporus CBS 130.89. The pulsed electric fields treatments were applied as describe in the example 3. One sample point was validated for a frequency of 8.6 Hz (treatment A). The temperature of the apple juice did not exceed 34° C., which implies a mild treatment. Even a more severe PEF treatment does not lead to fully inactivation of the spores of Talaromyces macrosporus.

Apple juice was inoculated with 104 spores/ml of apple juice. Subsquently to the PEF-treatment, the spores suspended in the apple juice were submitted to a heat treatment at 80° C. for 5 minutes in order to stimulate their germination.

All experiments were executed in duplo.

The samples were incubated at room temperature until fungal growth was visually observed (turbidity of the juice). Growth was observed after 3 days of incubation at 25° C.

### EXAMPLE 10

### The Antifungal Effect on Heat-Resistant Fungus Spores of a Combined Treatment: PEF with Natamycin in Apple Juice

This example describes the effect of natamycin combined with a Pulsed Electric Field treatment against the fungal strains described in example 8.

The chosen inoculation levels, natamycin concentrations and PEF treatments were as described in the examples 8 and 9. The natamycin can be added before or after the PEF treatment.

All experiments were executed in duplo.

All samples were incubated at room temperature until fungal growth was visually observed (turbidity of the juice). Growth was observed after 9 days of incubation at 25° C.

The above results clearly demonstrate that a combination of low natamycin concentration and an extremely mild PEF-treatment prevent the spoilage of apple juice by heat resistant fungus.

1. A pumpable material that has been subjected to a treatment that effects electroporation of any vegetative cells contained therein and which comprises an effective amount of an antifungal compound.

2. The pumpable material of claim 1, which is a food, feed containing liquid or a beverage.

3. The pumpable material of claim 1 wherein the method treatment is a pulsed electric field method.

4. The pumpable material of claim 1 where the antifungal compound is natamycin or sorbic acid.

5. The pumpable material of claim 1 further comprising an antibacterial agent.

6. The pumpable material of claim 6 wherein the antibacterial agent is nisin or lysozyme.

7. A method for treating a pumpable material, which comprises the steps of:
(a) subjecting the pumpable material to a treatment that effects electroporation of any vegetative cells contain therein; and
(b) adding an effective amount of an antifungal compound to pumpable material, said steps being performed in any order.

8. The method of claim 7 wherein the electroporation method treatment is a pulsed electric field method.

9. The method of claim 7 wherein the antifungal compound is natamycin or sorbic acid.

10. The method according to anyone of claim 7 wherein the antifungal compound is added to the pumpable material before the electroporation method.

11. The method of claim 7 further comprising adding an effective amount of antibacterial agent.

12. The method according to of claim 11 wherein the antibacterial agent is nisin or lysozyme.

13. A pumpable material obtainable by the method claim 7.

14-15. (canceled)

16. A liquid comprising sugars and proteins and/or free amino acids which is microbially stable for at least 30 days, free of sensory effect induced by a heat treatment and comprising an antifungal agent.

17. The liquid of claim 16 which is a fruit juice, lemonade, wine or beer.

18. The pumpable product of claim 1 which is a liquid.

19. The pumpable product of claim 18 which is a fruit juice, lemonade, wine or beer.

20. The pumpable product of claim 19 which is apple juice.

21. The liquid of claim 17 which is apple juice.

22. The method of claim 7 wherein the pumpable product is a liquid.
